(19) United States
(12) Patent Application Publication
(10) Pub. No.: US 2002/0007097 A1
(43) Pub. Date: Jan. 17, 2002

(54) Catalysts for heterogeneously catalyzed reactions

(76) Inventors: Christian Walsdorff, Ludwigshafen (DE); Ruprecht MeiBner, Weisenheim (DE); Klaus Harth, Altleiningen (DE)

Correspondence Address: Herbert B. Keil, KEIL & WEINKAUF, 1101 Connecticut Ave., N.W., Washington, DC, 20036, US

(21) Appl. No.: 09/847,298

(22) Filed: May 3, 2001

(30) Foreign Application Priority Data
May 19, 2000 (DE) 10024928.0

(51) Int. Cl. B01J 23/04; B01J 23/10; B01J 23/72; C07C 27/16

(52) U.S. Cl. 568/910; 502/341; 502/346; 502/344; 502/304; 502/302

(57) ABSTRACT

Catalysts for heterogeneously catalyzed reactions which comprise active components and a catalyst support comprising amounts of δ-Al₂O₃ which can be detected by X-ray diffractometry, in particular for the oxychlorination of ethylene to 1,2-dichloroethane.

The present invention relates to catalysts for heterogeneously catalyzed reactions having active components and a catalyst support which comprises amounts of δ-Al₂O₃ which can be detected by X-ray diffractometry.

EP-A-375 202 and U.S. Pat. No. 5,011,808 disclose oxychlorination catalysts comprising copper, potassium and magnesium on an ε-Al₂O₃ or y-Al₂O₃ support.

EP-A-931 587 discloses oxychlorination catalysts comprising a first layer of magnesium and a second layer of copper and optionally lithium on a γ-Al₂O₃ support.

EP-A-255 156 discloses oxychlorination catalysts comprising copper, magnesium and sodium or lithium on a γ-Al₂O₃ support.

U.S. Pat. No. 5,527,754 discloses oxychlorination catalysts comprising copper, magnesium and cesium or a mixture of cesium and potassium on an aluminum oxide. γ-Al₂O₃ is mentioned as being highly suitable.

However, these catalysts are still unsatisfactory.

It is an object of the present invention to provide supported oxychlorination catalysts having improved properties.

We have found that this object is achieved by novel and improved catalysts for heterogeneously catalyzed reactions which comprises active components and a catalyst support comprising amounts of δ-Al₂O₃ which can be detected by X-ray diffractometry.

δ-Al₂O₃ is commercially available or can be obtained by calcination of pseudoboehmite at temperatures of about 1000° C.

The catalyst supports generally have a BET surface area of from 80 to 250 g/m², preferably from 100 to 200 g/m², particularly preferably from 120 to 180 g/m², and a pore volume of from 0.2 to 1 cm³/g, preferably from 0.3 to 0.8 cm³/g, particularly preferably from 0.4 to 0.7 cm³/g.

δ-Al₂O₃ can be classified on the basis of its X-ray diffraction diagram. In “Aluminium Compounds”, G. Mac Zura, K. P. Goodboy and J. J. Koenig, Kirk-Othmer Encyclopedia of Technology, Volume 2, Third Edition (1978), pages 218 to 244, a review is given with ongoing references to the preparation and characterization of δ-Al₂O₃. δ-Al₂O₃ is commercially available.

Catalyst supports comprising amounts of δ-Al₂O₃ which can be detected by X-ray diffractometry generally comprise from 10 to 100% by weight of δ-Al₂O₃, preferably from 30 to 100% by weight of δ-Al₂O₃, particularly preferably from 50 to 100% by weight of δ-Al₂O₃, in particular from 60 to 100% by weight of δ-Al₂O₃. As a consequence of the preparation, residual constituents of γ-Al₂O₃ may also be present.

In addition to the catalyst support, the catalysts according to the invention also comprises active components. Suitable active components are from 1 to 15% by weight, preferably from 2 to 10% by weight, particularly preferably from 4 to 8% by weight, of copper, from 0.1 to 6% by weight, preferably from 0.2 to 4% by weight, particularly preferably from 0.2 to 3% by weight, in particular from 0.25 to 2% by weight, of an alkali metal, such as lithium, sodium, potassium, cesium, preferably potassium, from 0 to 5% by weight, preferably from 0.1 to 3% by weight, particularly preferably from 0.1 to 2% by weight, of an alkaline earth metal, such as calcium, magnesium, barium or strontium, preferably magnesium, of a rare-earth metal, such as cerium or lanthanum, or mixtures thereof.

Soluble salts are those which dissolve in water, a C₁- to C₄-alkanol, such as methanol, ethanol, propanol or butanol, a ketone, such as acetone, or an ester, such as methyl acetate or ethyl acetate, preferably in water.

Examples of suitable soluble salts are chlorides, nitrates, carbonates and acetates, preferably chlorides, nitrates and acetates, particularly preferably chlorides.

The catalysts according to the invention may also contain impurities from the δ-Al₂O₃ starting material, the pseudoboehmite, such as, for example, iron.

The catalysts according to the invention are advantageously obtained by impregnating the catalyst support and drying at from 80 to 250° C., preferably at from 90 to 200° C., particularly preferably at from 100 to 150° C. The impregnation can be carried out in one or more steps. The impregnation can preferably be carried out in one step and “dry”. “Dry” means that the concentration and amount of the impregnation solution are matched to the water absorption capacity of the support used in such a way that virtually all the impregnation solution is taken up by the support material and the entire support material is impregnated uniformly. If desired, acids, such as inorganic acids, for example hydrochloric acid or nitric acid, or organic acids, such as carboxylic acids, for example acetic acid, preferably hydrochloric acid, or oxidants, such as hydrogen peroxide, may also be added to the impregnation solution in order, for example, to obtain a clear impregnation solution and to simplify the impregnation. For use as a fluidized-bed catalyst, a pulverulent support is preferably impregnated, while for use as a fixed-bed catalyst, moldings of the support material which have been shaped and, if desired, calcined in advance are preferably employed.

The catalysts can be used in powder form for use in fluidized-bed processes or as moldings for use in fixed-bed processes. On using fixed-bed processes, preference is given to supports having a low-pressure-loss geometry and having a high geometrical surface area, such as, for example, rings or hollow cylinders having one or more holes. In order to achieve better mechanical stability or pore structure of such moldings, use can be made of δ-Al₂O₃ or alternatively a compound which can be converted into δ-Al₂O₃ by calcination after shaping of the support element, for example pseudoboehmite.

The catalysts according to the invention are distinguished over catalysts prepared in a conventional manner using γ-Al₂O₃ with the same percentage content of active material by significantly better selectivity at the same time as comparable activity.

The catalysts according to the invention are suitable for heterogeneously catalyzed reactions, such as exothermic gas-phase reactions, for example oxidation reactions, particularly for oxychlorination, in particular for the oxychlorination of ethylene to 1,2-dichloroethane (ethylene dichloride, and oxidation reactions).

The oxychlorination, in particular that of ethylene to give 1,2-dichloroethane (ethylene dichloride), can be carried out at temperatures of from 150 to 400° C., preferably from 170 to 350° C., particularly preferably from 200 to 300° C., and a pressure of from 1 to 10 bar, preferably from 1 to 6 bar, particularly preferably from 1 to 4 bar.

### EXAMPLES

The catalysts were tested in powder form in a laboratory fluidized-bed reactor. Example and comparative catalysts were 5 tested under the same conditions in the same test apparatus. The laboratory reactor had a diameter of 2.5 cm and was temperature-controlled by a stirred oil bath in a reactor double wall. The temperature of the fluidized bed was measured and controlled via a thermocouple inserted into a thermosleeve in the fluidized bed. In each case, 90 g of the catalysts were charged with a stoichiometric feed of one mole of hydrogen chloride, half a mole of ethylene and a quarter of a mole of oxygen in the form of air, and tested at a pressure of 1.2 bar and temperatures of 225° C., 245° C. and 265° C. Under these conditions, the relatively short residence time (about 7 s) meant that full conversion was not achieved.

The catalysts according to the invention were obtained by impregnating Puralox® SCCa 5/150 (a δ-Al₂O₃ from Condea) with a clear solution of the promoters in water. The comparative catalysts were prepared on a Puralox® SCCa 5/200 support (a γ-Al₂O₃ from Condea). Various catalysts according to the invention and comparative catalysts were prepared and tested with the same promoter composition in each case.

### Example 1

34.93 g of CUCl₂.2H₂O, 15.84 g of MgCl₂.6H₂O and 1.80 g of KCl were dissolved in 156 ml of water. This solution was impregnated, with mixing, onto 200 g of Puralox® SCCa 5/150. The impregnated support was left to stand at room temperature for 1 hour and subsequently dried at 110° C. for 16 hours under a stream of nitrogen.

TABLE 1
Test results for the catalyst from Example 1

| | Ethyl- | | | | Ethylene |
|---|---|---|---|---|---|
| | ene | Ethylene | | Chlorinated | dichlo- |
| Tempera- | conver- | dichloride | CO + CO₂ | hydrocarbons | ride |
| ture | sion | selectivity | selectivity | selectivity | yield |
| 225° C. | 61.1% | 99.6% | 0.14% | 0.17% | 60.9% |
| 245° C. | 77.3% | 99.0% | 0.58% | 0.35% | 76.6% |
| 265° C. | 82.9% | 96.8% | 2.34% | 0.82% | 80.3% |

*Total of ethyl chloride, 1,1,2-trichloroethane, 1,1,2,2-tetrachloroethane, 1,1-dichloroethane, dichloroethene (isomers), chloral, vinyl chloride, tetrachloromethane and chloroform

### Comparative Example 1

34.93 g of CUCl₂.2H₂O, 15.84 g of MgCl₂.6H₂O and 1.80 g of KCl ere dissolved in 160 ml of water. This solution was impregnated, with mixing, onto 200 g of Puralox® SCCa 5/200. The impregnated support was left to stand at room temperature for 1 hour and subsequently dried at 110° C. for 16 hours under a stream of nitrogen.

TABLE 2
Test results for the catalyst from Comparative Example 1

| | Ethyl- | | | | Ethylene |
|---|---|---|---|---|---|
| | ene | Ethylene | | Chlorinated | dichlo- |
| Tempera- | conver- | dichloride | CO + CO₂ | hydrocarbons | ride |
| ture | sion | selectivity | selectivity | selectivity | yield |
| 225° C. | 65.9% | 99.4% | 0.39% | 0.2% | 65.5% |
| 245° C. | 78.5% | 98.3% | 1.18% | 0.44% | 77.2% |
| 265° C. | 83.5% | 95.2% | 3.72% | 1.05% | 79.6% |

*Total of ethyl chloride, 1,1,2-trichloroethane, 1,1,2,2-tetrachloroethane, 1,1-dichloroethane, dichloroethene (isomers), chloral, vinyl chloride, tetrachloromethane and chloroform

We claim:

1. A catalyst for heterogeneously catalyzed reactions, which comprises active components and a catalyst support comprising amounts of δ-Al₂O₃ which can be detected by X-ray diffractometry.

2. A catalyst for heterogeneously catalyzed reactions as claimed in claim 1, wherein the catalyst support comprises from 10 to 100% by weight of δ-Al₂O₃.

3. A catalyst for heterogeneously catalyzed reactions as claimed in either of claims 1 and 2, wherein the active components employed are from 1 to 15% by weight of copper, from 0.1 to 6% by weight of alkali metals, from 0 to 5% by weight of alkaline earth metals, rare-earth metals or mixtures thereof.

4. A catalyst for heterogeneously catalyzed reactions as claimed in claim 1, prepared by impregnating a shaped δ-Al₂O₃-containing support having a BET surface area of from 80 to 250 g/m² with salts of copper, alkali metals and, if desired, alkaline earth metals, rare-earth metals or mixtures thereof.

5. A process for the preparation of a catalyst for heterogeneously catalyzed reactions as claimed in one of claims 1 to 3, which comprises impregnating the δ-Al₂O₃-containing support with salts of copper, alkali metals and, if desired, alkaline earth metals, rare-earth metals or mixtures thereof, separately from one another or together, if desired with the addition of acids or oxidants.

6. A process for the preparation of a catalyst for heterogeneously catalyzed reactions as claimed in claim 5, wherein the salts employed are chlorides.

7. The use of a catalyst for heterogeneously catalyzed reactions as claimed in one of claims 1 to 4 for exothermic gas-phase reactions.

8. The use of a catalyst for heterogeneously catalyzed reactions as claimed in one of claims 1 to 4 for oxychlorination reactions.

9. The use of a catalyst for heterogeneously catalyzed reactions as claimed in one of claims 1 to 4 for the oxychlorination of ethylene to 1,2-dichloroethane.

10. A process for the preparation of 1,2-dichloroethane, which comprises reacting ethylene with hydrogen chloride and air or oxygen in the presence of a catalyst as claimed in one of claims 1 to 4 at a temperature of from 150 to 400° C. and a pressure of from 1 to 10 bar.

11. The use of a catalyst for heterogeneously catalyzed reactions as claimed in one of claims 1 to 4 for partial oxidation reactions.
